(19) United States
(12) Patent Application Publication
(10) Pub. No.: US 2014/0128292 A1
(43) Pub. Date: May 8, 2014

(54) METHODS FOR IMPROVING LIGATION STEPS TO MINIMIZE BIAS DURING PRODUCTION OF LIBRARIES FOR MASSIVELY PARALLEL SEQUENCING

(71) Applicants: Masoud M. Toloue, Austin, TX (US); Jason Dickson, Austin, TX (US); Prachi Nakashe, Fort Worth, TX (US); Marianna Goldrick, Austin, TX (US)

(72) Inventors: Masoud M. Toloue, Austin, TX (US); Jason Dickson, Austin, TX (US); Prachi Nakashe, Fort Worth, TX (US); Marianna Goldrick, Austin, TX (US)

(73) Assignee: BIOO SCIENTIFIC CORPORATION, Austin, TX (US)

(21) Appl. No.: 14/040,133

(22) Filed: Sep. 27, 2013

Related U.S. Application Data
(60) Provisional application No. 61/706,451, filed on Sep. 27, 2012.

(51) Int. Cl. C12N 15/10

(52) U.S. Cl. CPC C12N 15/1096
USPC 506/26

(57) ABSTRACT

Described herein is a thermostable enzyme capable of efficient ligation of two oligomers at high temperature. The embodiments herein have led to the development of an optimized ligation step used in library preparation for sequencing reactions.

### PRIORITY CLAIM

This application claims the benefit of U.S. Provisional Application No. 61/706,451 filed on Sep. 27, 2012.

### BACKGROUND OF THE INVENTION

1. Field of the Invention

The present invention relates to the field of nucleic acid sequence determination, and novel approaches to sequencing small RNA libraries in a massive and high-throughput manner.

2. Description of the Relevant Art

“Sequencing” is the term used to describe the process of determining the order of nucleotides in polynucleotide molecules such as genomic DNA and messenger RNA. The technology for sequencing has evolved over the several decades since it was first invented. Initially, sequencing required clonal amplification of individual target molecules in plasmid or phage vectors, and the resulting templates were then sequenced in individual reactions and analyzed in separate lanes of high resolution polyacrylamide gels or, after the invention of automated sequencing, in separate channels or capillaries.

More recently, newer sequencing technologies rely on simultaneous amplification of complex populations of DNA or RNA targets using the polymerase chain reaction (PCR). The complex populations may comprise fragments of DNA derived from whole genomes of cells or tissues, or the entire populations of RNAs (“transcriptomes”) present in cells or tissues. The amplified populations are then sequenced in parallel, enabling much higher throughput in acquisition of sequencing data, and at a much reduced cost. The newer methods are often referred to as “massively parallel sequencing” or “Next Generation Sequencing” (NGS). NGS approaches for sequencing generally involve acquiring information as short “reads” of several dozen to at most, a few hundred bases, and thus present a higher demand for bioinformatics resources to assemble the reads into interpretable data. Methods to increase the quality of the short reads obtained using NGS technology are useful to facilitate assembly of the data into useful form. Advantages of NGS approaches as compared to original sequencing methods include higher sensitivity for detecting low-abundance RNAs, opportunities to discover new small RNAs, and ability to use multiplex approaches to allow multiple samples to be assessed in a single experiment.

The amplified populations of complex DNA or RNA molecules are often referred to as “libraries”, and are produced by using the primary genetic material (as may be obtained for example by extraction of DNA or RNA from malignant tumor cells or from healthy normal cells) as input for a series of enzymatic modifications catalyzed by enzymes commonly used for molecular biology applications. Examples of such enzymes are RNA and DNA polymerases, RNA and DNA ligases, reverse transcriptases, thermostable DNA polymerases, etc. The enzymatic steps serve to introduce specific synthetic oligonucleotide sequences into the primary target material, said sequences being necessary for exponentially increasing the number of target molecules by PCR (known as “amplifying the library”) to levels required for sequencing, and for adding sequences required for associating the library with the NGS instrument. The new sequencing technologies have enabled unprecedented ability to acquire genomic data, for example to determine sequences of entire genomes, and to determine the entire RNA output (known as “global expression profiling”) of particular cells and tissues. RNA output can refer to traditional mRNAs that reflect protein-coding sequences, or non-coding RNAs including microRNAs and other small RNAs, as well as long non-coding RNAs.

The library amplification step used to create NGS libraries is typically carried out using PCR. To create the recognition sites for binding the Forward and Reverse PCR primers, and to introduce sequences needed for associating the targets with the NGS sequencing instruments, oligonucleotide “adapters” are appended to the target sequences. The adapters are typically appended sequentially to both ends of target molecules using ligase enzymes. For example, T4 DNA ligase can be used to catalyze addition of DNA oligonucleotides to target DNAs via formation of covalent phosphodiester bonds. Other ligases that have been used to create NGS libraries include various RNA ligases. For example, a truncated form of T4 RNA ligase 2 has been used in creating NGS libraries. Truncated T4 RNA Ligase 2 is a member of a family of RNA ligases that are defined by essential signature residues in the C-terminal domain. Mutational analysis of T4 RNA Ligase 2 has identified several amino acids that are essential for strand joining (Ho and Shuman (2002); Yin et al. (2003), the truncated version of which comprises an autonomous adenylyltransferase/AppRNA ligase domain (Ho et al. (2004) Cell 12:327. Optimum pH conditions of the adenylyltransferase activity of full length T4 RNA Ligase 2 and truncated T4 RNA Ligase 2 (pH 6.5 and pH 9-9.5 respectively) are prior art described in Ho et al. (2004) Cell 12:327.

During the amplification step used to produce NGS libraries, it is desirable to preserve the original relative levels of the different target molecules in the amplified product. Examples of target molecules are genomic DNA fragments, cDNAs produced from mRNAs, and small RNAs such as microRNAs. Maintaining relative levels of target molecules allows the library to be used to derive quantitative information about differences between levels of targets within a sample and between samples. For example, it is desirable to determine whether relative expression of specific microRNAs differs between malignant cells and non-malignant cells.

Intrinsic differences exist in the ability of different targets to serve as substrate for the enzymatic steps (including ligation, reverse transcription and PCR amplification) that are used to create amplified libraries. The intrinsic differences are due to sequence differences between target molecules. These sequence differences lead to uneven amplification of the different targets, such that unwanted “bias” is introduced into the NGS library. Bias refers to differences in relative levels of target DNAs or RNAs in the NGS libraries, as compared to the relative levels of the targets in the unamplified complex starting population of DNA or RNA sequences. Methods to reduce bias during NGS library construction are useful to facilitate quantitative analysis of the starting population, for example to discover microRNA expression differences between normal and malignant cells.

An important feature of target molecules that can lead to bias in NGS libraries is the presence of sequences capable of forming stable secondary structures. In the context of single-stranded RNA, secondary structure refers to regions of nucleotides within an RNA molecule that interact to form more complex shapes (compared to a linear polynucleotide structure); such interactions are commonly based on hydrogen bonding of complementary base pairs. The presence of secondary structure in target RNA molecules generally interferes with the enzymatic steps used to create NGS libraries. Enzymatic ligation is especially affected and such bias leads to over representation and under representation of individual RNA molecules in the population.

Bias introduced during the ligation steps of NGS library production can also be due to intrinsic target sequence features, independent of their ability to form secondary structures. Particular RNA ligases are known to have inherent biases for ligating targets with particular base compositions. For example, T4 RNA Ligase 1, used to ligate the 5′ adapter to sample RNA has been shown to have strong sequence preference toward adenine (Romaniuk et al. 1982). The reason for this bias is thought to relate to the observation that bacteria under viral stress nick their tRNAs to block the translation of mRNA into protein. T4 phage (from which T4 Ligase 1 is derived) uses RNA ligase to repair the nick. Since these nicks are made at specific sequences in the tRNAs, T4 RNA Ligase 1 has likely evolved sequence specificity to efficiently repair the nicks.

Small RNA sequencing using NGS technology is now a standard for determining global profiles of small RNA populations. MicroRNAs (miRNAs) are a specific subset of small RNAs which have garnered much interest in recent years. Changes in miRNA expression have been shown to be associated with a variety of normal physiological processes as well as diseases including cancer. Studies have already shown that miRNAs may provide useful markers for the development of disease diagnostic and prognostic assays. NGS technologies are in principle very well suited for high-throughput sequencing of small non-coding RNAs. Despite this promise, NGS sequencing data is often plagued by bias, which compromises the interpretation of data within samples and between samples.

Typically 15-45 nucleotides in length, small RNAs play important roles in the regulation of protein-coding genes and in regulation of other features of the genome. Small non coding RNAs (ncRNAs) have been classified as microRNA (miRNA), short interfering RNA (siRNA), piwi RNA (piRNA), and small nucleolar RNA (snoRNA). Complex RNA extracted from biological sources also contains longer non-coding RNAs (long ncRNAs). Most of the ncRNAs in the genome have yet to be discovered and validated for function. Evidence has shown that many ncRNAs play key roles in processes such as cellular differentiation, cell death, and cell metabolism. Several groups have reported methods for cloning miRNAs from primary RNA sources (Berezikov et al. (2006) Nature Genetics 38:S2; Cummins et al. (2006) Proc. Natl. Acad. Sci. 103: 3687; Elbashir et al. (2001) Genes and Development 15:188; Lau et al. (2001) Science 294:858; Pfeffer et al. (2003) Curr Protocols Mol Bio 26.4.1). In order for small RNAs to be isolated and sequenced, a sequential series of enzymatic steps including ligation, reverse transcription, and amplification are carried out to generate the NGS libraries, i.e. the material to be analyzed on a NGS sequencing instrument.

Preparation of samples for next-gen sequencing of small RNAs generally involves an initial step of extracting total RNA, usually followed by an enrichment step to eliminate large RNAs greater than ˜100 bases, and sometimes an additional fractionation step to recover only RNAs in the size-range of microRNAs (˜15-30 bases). With the RNA in hand, the next step is to add common oligonucleotide sequences (“linkers”) to the 5′ and 3′ ends of the RNA population, in order to provide binding sites for Forward and Reverse PCR primers, so that the RNA population can be amplified and modified to include sequences complementary to capture oligos (“adapters”) used by the sequencing instrument to capture the templates into flow cells or onto slides as appropriate for the sequencing platform to be utilized. Two purification steps using high-resolution polyacrylamide gels are typically carried out during the linker addition steps used to create the small RNA library. The first gel purification step is used to recover RNAs after ligation of the first linker, which is usually the 3′ linker, and the second gel purification step is used to recover the final product, after ligation of the second linker (i.e. the 5′ linker). Gel purification is needed to remove components of the ligation reaction buffers and unwanted side products that could interfere with the subsequent steps, including PCR amplification of the small RNA library and the sequencing reaction itself. Examples of unwanted side products are 5′/3′ linkers that are ligated to each other without an intervening target RNA, and target RNAs to which only a single linker has been added. Gel purification is a time-consuming, labor-intensive process that can lead to loss of material. Gel purification is especially problematic in the context of small RNA library construction, since the target molecules are too small (typically in the size range of ˜60-100 bases) to be easily stained, resolved, and visualized on polyacrylamide gels. Also, the size separation between the target products and unwanted side products is only 20-30 nucleotides, making it tedious to carry out the extraction. It would be desirable to develop methods that eliminate the requirement for gel purification during small RNA library construction. This disclosure describes an approach to accomplish that goal.

### SUMMARY OF THE INVENTION

In one embodiment, a method of producing a library includes: obtaining a population of RNA molecules; ligating a 3′ adapter oligonucleotide containing RNA bases, DNA bases, and/or synthetic bases and/or modified and/or randomized bases to the 3′ end of the population of RNA molecules, wherein a thermostable ligase is used to catalyze the ligation reaction and wherein the 3′ adapter oligonucleotide ligation reaction is carried out at a temperature greater than 40° C.; ligating a 5′ RNA oligonucleotide adapter to the population of RNA molecules, wherein a thermostable ligase is used to catalyze the ligation reaction and wherein the 5′ adapter oligonucleotide ligation reaction is carried out at a temperature greater than 40° C.; and converting the population of 3′/5′ ligated RNA molecules to complementary DNA (cDNA) molecules using reverse transcription. Optionally, the resulting cDNA molecules are amplified by polymerase chain reaction. Optionally the population of 3′/5′ ligated RNA molecules are purified prior to further reaction (e.g., prior to reverse transcription or PCR.

In another embodiment, a method of producing a library includes: obtaining a population of RNA molecules and/or DNA molecules; ligating a 3′ adapter oligonucleotide to the 3′ end of the population of RNA molecules and/or DNA molecules, wherein a thermostable ligase is used to catalyze the ligation reaction and wherein the 3′ adapter oligonucleotide ligation reaction is carried out at a temperature greater than 40° C.; converting the population of 3′ ligated RNA molecules and/or DNA molecules to complementary DNA (cDNA) molecules using reverse transcription; intramolecularly ligating the resulting cDNA products; and cleaving the resulting intramolecularly ligated cDNA using a targeted single-stranded DNA endonuclease to form linearized cDNA products. Optionally, the resulting linearized cDNA molecules are amplified by polymerase chain reaction. Optionally the population of 3′ ligated RNA and/or DNA molecules are purified prior to further reaction (e.g., prior to reverse transcription, intramolecular ligation, or PCR.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic diagram of the process steps for Method 1; and

FIG. 2 depicts a schematic diagram of the process steps for Method 2.

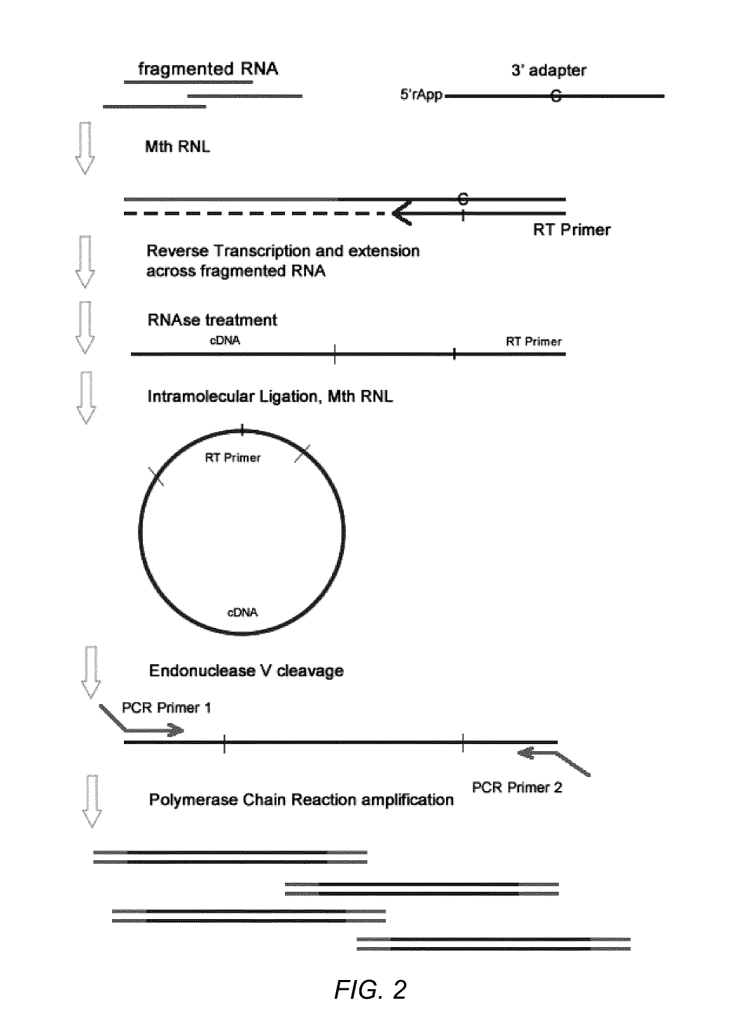

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

A particular benefit of the methods described herein is that the methods reduce bias in NGS libraries. In the context of this invention, “bias” refers to alterations in the proportional number of reads of specific sequences during massively parallel sequencing of complex mixtures of target RNA or DNA molecules, compared to the true relative levels of the specific RNA or DNA molecules that are actually present in the complex mixture. Bias can be due to many factors, for example intrinsic differences in the efficiency with which the different sequences are able to serve as templates for producing amplified sequencing templates for massively parallel sequencing.

In one embodiment, a method of producing NGS libraries comprises inhibiting the tendency for single stranded RNA to form secondary structures by adjusting reaction conditions such as the temperature and ionic strength. Higher temperatures and lower ionic strength tend to reduce secondary structure formation. Carrying out the ligation steps used to produce NGS libraries at higher temperatures can be beneficial for minimizing secondary structure in the target molecules, thereby minimizing bias in the resulting libraries. The method described herein minimizes bias in NGS libraries by allowing ligation steps to be carried out at elevated temperatures, compared to temperatures conventionally used for the ligation steps. Elevated temperatures which inhibit the formation of second structures include carrying out the ligation at temperatures above about 37° C.; above about 40° C.; above about 50° C.; or above 60° C. The ligase reaction, however, is run at a temperature that is less than the temperature that will inactivate or decompose the ligation enzyme (typically less than 100° C.). In some embodiments, the ligation reaction is carried out at a temperature of between about 37° C. and about 75° C. or between about at 50° C. to about 65° C. The method is envisioned to be especially useful in the context of creating NGS libraries for small RNA sequencing. The ligation reaction is run for a time effective to carry out the ligation reaction to completion. Typical reaction times are between about 10 minutes and 120 minutes at elevated temperatures.

Another advantage of the methods described herein is the use of alternative, non-traditional ligase having less inherent bias for ligation of specific sequences found in the target RNA. One of the most critical steps in this type of small RNA sequencing is the ligation step. A particular feature of the instant invention is use of a novel ligation reaction buffer composition in conjunction with a prolonged time period of incubation, which results in the advantages of significantly increasing the yield of NGS library generated from the primary small RNA, compared to yields of small RNA libraries produced using standard methods.

We have discovered that a particular ligase, namely Mth RNA Ligase (MthRNL) produced by the thermophilic archaebacteria Methanobacterium thermoautotrophicum, is advantageous for use in creating libraries for NGS, said libraries having reduced bias compared to NGS libraries made using alternative ligases that have traditionally been used to make NGS libraries. In some embodiments, Mth RNA ligase is purified from a recombinant source. Mutant Mth RNA Ligase may be used. Examples of Mth RNA Ligase mutants include, but are not limited to: Mth RNA Ligase may be Mth RNA Ligase mutant K97A, Mth RNA Ligase mutant K246A, Mth RNA Ligase single mutant of any amino acids associated with the adenylyltransferase Motifs I through V, Mth RNA Ligase double mutant of any amino acids associated with the adenylyltransferase Motifs I through V, and Mth RNA Ligase triple mutant of any amino acids associated with the adenylyltransferase Motifs I through V.

The ligation steps disclosed herein are particularly useful for methods such as sequencing, high-throughput sequencing, barcoded sequencing (multiplex analysis), small RNA capture, cloning and quantitative PCR. In some embodiments, the ligation reactions disclosed herein are used on a population of RNA molecules that includes small RNAs ranging in size from about 15 bases to about 100 bases.

The ligation reactions (for both the 3′ and 5′ ends) are carried out in a ligation reaction buffer. The ligation reaction buffer may include: magnesium chloride at a concentration of between about 1 mM to about 50 mM; dithiothreitol at a concentration ranging from about 1 mM to about 50 mM; and Tris-HCl at a concentration ranging from about 1 mM to about 100 mM. The pH of the ligation reaction buffer may be between about 5 to about 10. In one embodiment, performing a ligation reaction in a ligation reaction buffer of 50 mM TrisHCl, pH 7.5, 10 mM MgCl₂ and 1 mM DTT, is significantly more efficient than reactions performed in other ligation reaction buffers, allowing maximum ligation of linker to sample; allows for greater sequencing coverage per reaction; results in less sequence bias due to uneven ligation compared to typical current ligase buffer conditions; and results in higher ligation binding of adenylated adapters. Furthermore, it was found that magnesium chloride concentration has a significant effect on the efficiency of the ligation reaction. A ligation reaction buffer having a concentration of MgCl₂ in a range of about 10 mM MgCl₂ to about 30 mM MgCl₂ was found to be the most optimal for the truncated T4 RNA Ligase 2 enzyme to ligate two oligomers.

In certain exemplary embodiments, a method for sequencing multiple nucleic acid sequences at once is provided. The method involves the use of an adenylated adapter in the presence of ligase and in the absence of ATP, allowing the adenylated oligonucleotide to bind to the 3′ or 5′ end of the small RNA or nucleic acid sample to form a ligation product. The second ligation is performed with a ligase that requires ATP on the 5′ or 3′ end and establishes a second tag on the other end of the small RNA or nucleic acid sample. The ligated products may then be sequenced directly, reverse transcribed and amplified for sequencing or sequenced after a reverse transcription step directly. The oligonucleotide may be present in an amount ranging from 1 ng-50 μg. If an adenylated oligonucleotide is used, the concentration may be between about 1 μM to about 1000 μM during the ligation reaction.

The oligonucleotide ligation reaction may be performed in the presence of polyethylene glycol (PEG) having a molecular weight of between about 4000 to about 8000, which is present at a concentration ranging from 0.1% to about 90%.

After the 3′ and/or 5′ ends of the population of RNA are ligated with adapter oligonucleotides, the resulting 3′/5′ ligated oligonucleotides may be converted to complementary DNA (cDNA) molecules using reverse transcription. The cDNA molecules may, optionally, be amplified by polymerase chain reaction (PCR). Prior to reverse transcription or PCR the 3′/5′ ligated oligonucleotides may be purified. Methods of purification of the 3′/5′ ligated oligonucleotides include, but are not limited to, column, magnetic bead, or precipitation-based purification. Purification of the 3′/5′ ligated oligonucleotides removes excess buffers and enzymes remaining from the ligation steps.

In an alternate embodiment, after the 3′ end of ae population of RNA and/or DNA molecules is ligated with adapter oligonucleotides, the resulting 3′ ligated oligonucleotides may be converted to complementary DNA (cDNA) molecules using reverse transcription. The cDNA molecules are intramolecularly ligated, then cleaved using a targeted single-stranded DNA endonuclease (e.g., Endonuclease V) to form linearized cDNA products. Intramolecular ligation may be performed using Mth RNA ligase or Mh RNA ligase mutants.

In certain exemplary embodiments, a method for highly efficient ligation is disclosed using Mth RNA Ligase wherein the unit activity is defined as: 200 units of the enzyme required to give 80% ligation of a 31-mer RNA to the 5′ pre-adenylated end of a 17-mer DNA in a total reaction volume of 10 μL in 1 hour at 37° C.

In certain exemplary embodiments, a method for highly efficient ligation using Mth RNA Ligase with enhanced buffer conditions is provided. The buffer of the ligase efficiently allows the binding of adenylated oligonucleotides to the ends of sample nucleic acids or binding of oligonucleotides to the ends of sample nucleic acids in the presence of ATP in a much more efficient manner than published descriptions using different ligases. This allows maximum ligation of linker to sample, allowing for greater sequencing coverage per reaction, less sequence bias due to uneven ligation compared to typical current ligase buffer conditions, and higher ligation binding of adenylated adapters.

In certain exemplary embodiments, a new method for ligation is disclosed that can be used in 3′ adapter and 5′ adapter steps for sequencing of small RNAs or for a single ligation of a combined 3′ and 5′ adapter with or without modifications.

In certain exemplary embodiments, a new method for ligation is disclosed where T4 RNA Ligases are mixed with Mth RNA Ligase at varying ratios and used for adapter ligation.

In certain exemplary embodiments, a new method for ligation is disclosed where Mth RNA Ligase has been mutated to enhance ligation binding, or mutated to eliminate de-adenylation or adenylation to enhance adapter ligation.

In certain exemplary embodiments, a method for intramolecular ligation is disclosed where Mth RNA ligase is utilized, wherein its thermophilic qualilites enhance the formation of ssDNA and ssRNA loops.

In certain exemplary embodiments, a new library preparation method for RNA or DNA significantly shortens the library preparation procedure, reduces ligation steps and reduces sequence bias. The resulting libraries may be used for cloning, quantitative PCR, sequencing, high throughput sequencing tag labeling, barcoding and/or multiplexing multiple samples simultaneously. For example a library generated from small RNA molecules may be used to discover, profile and sequence non coding RNAs (ncRNAs), wherein ncRNAs include the microRNA (miRNA), piwi RNA (piRNA), small nucleolar RNA (snoRNA) and long ncRNAs.

The principles of the disclosed methods may be applied to enhance the ligation step and allow visualization of the ligation of two oligomers on a gel for the purposes of biological study of short or long strands of oligonucleotide. As used herein, a oligomer can refer to a single stranded or double stranded RNA, DNA, RNA/DNA hybrid consisting of anywhere from 2-1000 nucleotides. The methods also provide a highly efficient strategy to ligate a known strand of oligomer to an unknown strand of oligomer for the purpose of capture, cloning, quantitative PCR, sequencing and high-throughput sequencing applications. The methods allow users to increase sequencing yield, which is dependent on the efficiency with which known oligomers ligate to unknown species. Such a ligation of an oligonucleotide with enhanced buffer and Mth RNA Ligase does not require ATP, but may be performed using ATP and can be ligated to microRNA, siRNA, snoRNA, ssDNA and similar oligonucleotides from biological or synthetic samples in solution or on a solid support surface. Subsequent to ligation, samples can be reverse transcribed, amplified, and precipitated for use in capture or sequencing experiments. The methods described herein allow for sequencing on several platforms including Illumina, Solexa, Roche 454, SOliD, Helicos, Pacific Bio, PGM, Ion Torrent, Proton, Polonator and other similar platforms.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Method 1—Elevated Temperature Ligation

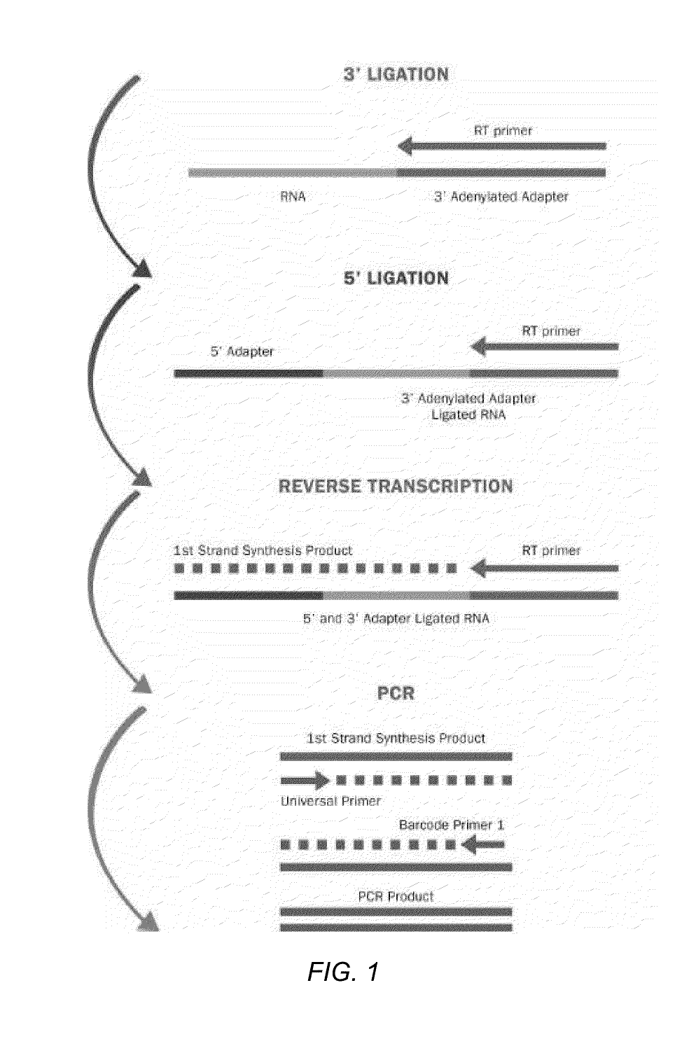

FIG. 1 depicts a schematic diagram of the process steps for Method 1.

### Step 1-3′ Adapter Ligation

### Materials:

0.05-25 μM 3′ adapters (Bioo Scientific)
10×Mth RNA Ligase buffer (Bioo Scientific)

### Mth RNA Ligase (Bioo Scientific)

RNA (0.1-100 μg of total RNA or isolated small RNA) (Bioo Scientific Cat. #5155-5182)

### 50% PEG (MW 4000 to MW 8000)

### RNase Inhibitor (Promega)

### Nuclease-Free Water (Bioo Scientific Cat. #801001)

1. Combine the following separately for EACH adapter:

| | | |
|---|---|---|
| 2-3 | μL | RNA (total RNA or isolated small RNA may be used) |
| 1 | μL | 3′ adapter (0.05-25 μM) |
| | | Heat at 70° C.-95° C. for 30 seconds to 2 minutes then |
| | | immediately place on ice. |
| 1 | μL | 10X AIR ™ Ligase buffer/10X Mth RNA Ligase |
| | | buffer |
| 2.4 | μL | 50% PEG (MW 4000/MW 8000) |
| 1 | μL | RNase Inhibitor |
| 2-3 | μL | Nuclease-Free Water |
| 10 | μL | TOTAL |

3. Add to each:
   1 μL AIR Ligase/Mth RNA Ligase
4. Incubate at 60° C.-75° C. for 1-2 hours.
5. Heat inactivate at 85° C.-95° C. for 5-15 minutes.

At this stage, proceed to step 2 or alternatively 2-100 μM RT primer can be annealed by heating at 70° C. for 5 minutes, then to 37° C. for 30 minutes, and then to 25° C. for 15 minutes. Annealing RT primer at this step helps reduce adapter dimer formation. Another alternative is to perform gel isolation of the 3′ adapter ligated RNA from the excess 3′ adapter by running the sample on a denaturing or non-denaturing polyacrylamide gel.

### Step 2—5′ Adapter Ligation

### Materials:

3′ adapter ligated RNA (from step 1)
0.05-20 μM 5′ adapter

### 10 mM ATP

Mth RNA Ligase/T4 RNA ligase 1
1. Heat 1 μL (for each reaction) of the 5′ adapter at 70° C. for 2 minutes and immediately place on ice.
2. Combine:

| | |
|---|---|
| 10 μL | 3′ adapter ligated RNA |
| 1 μL | 10 mM ATP |
| 1 μL | 5′ adapter (0.05-20 μM) |
| 1 μL | Mth RNA Ligase or T4 RNA ligase 1 |

3. Incubate at 20-65° C. for 1-2 hours
   At this stage, adapter-ligated RNA (from step 2) can be pooled with other RNA ligated barcodes. Alternatively, each RNA ligated barcode can proceed through the next steps individually and be pooled after step 6. Another alternative step is to carry out a column, magnetic beads, or precipitation-based cleanup method, and then proceed to Step 3. The cleanup gets rid of the excess buffers and enzymes from steps 1 and 2 that can inhibit steps 3 and 4.

### Step 3—Reverse Transcription—1^{st} Strand Synthesis

### Materials:

10×RT buffer (Bioo Scientific Cat. #521002)
MMuLV Reverse Transcriptase (Bioo Scientific Cat. #521002)/equivalent Reverse Transcriptase enzyme
12.5 mM dNTPs (Bioo Scientific Cat. #370601)

### 100 mM DTT

### RNase Inhibitor

### Nuclease-Free Water (Bioo Scientific Cat. #801001)

5′ and 3′ ligated RNA (13-14 μL) (from steps 1 and 2)
2-210 μM RT primer
1. Add 13 μL of 5′ and 3′ adapter ligated RNA to 1 μL RT primer (2-100 μM) if RT primer hasn't been annealed between step 1 and 2.
2. Incubate at 70° for 2 minutes and immediately place on ice
3. Add to each reaction

| | |
|---|---|
| 1 μL | 10X RT buffer |
| 0.5 μL | 12.5 mM dNTPs |
| 1 μL | 100 mM DTT |
| 0.5 μL | RNase Inhibitor |
| 1 μL | MMuLV Reverse Transcriptase/ |
| | equivalent Revese Transcriptase enzyme |
| 3 μL | Nuclease-Free Water |

4. Incubate at 44° C.-55° C. for 1-2 hours.
   Step 4 —cDNA Synthesis

### Materials:

### 10-50 μM PCR Primer 1

### 10-50 μM PCR Primer 2

### 5× DuroTaq PCR Master Mix (Bioo Scientific Cat. #370201)

### Nuclease-Free Water (Bioo Scientific Cat. #801001)

1^{st} strand synthesis product (10 μL)
1. Add to each 1^{st} strand synthesis reaction (10 μL)

| | |
|---|---|
| 18 μL | Nuclease-Free Water |
| 10 μL | 5x DuroTaq PCR Master Mix Mix/equivalent |
| | thermostable Polymerase enzyme |
| 1 μL | PCR Primer 1 |
| 1 μL | PCR Primer 2 |

2. Amplify

| | | | |
|---|---|---|---|
| 30 | sec | 95° C.-98° C. | |
| 10-15 | sec | 95° C.-98° C. | Repeat 5-25 cycles |
| 30-60 | sec | 55° C.-65° C. | |
| 15-30 | sec | 72° C. | |
| 10 | min | 72° C. | |

### Step 5—Purification

### Materials:

### 5-15% TBE gel

1×TBE buffer
Low molecular weight ladder

### Loading dye

1. Load samples with loading buffer into a 5-15% TBE gel
2. Run at 200 volts for 30-60 min
3. Cut the band corresponding to ˜150 nucleotides in length (do NOT cut out the 120 nucleotide band)
   Step 6—cDNA Purification

### Materials:

### 1×Tris-EDTA, pH 7.5 (TE)

### 3 M NaOAc

### 95% Ethanol

### 70% Ethanol

### Nuclease-Free Water

1. Shred the gel pieces and then soak them in 1×TE for at least 3 hours or overnight at room temperature.
2. Add 1/40^{th} volume 3 M NaOAc, 1/100^{th} volume glycogen and 4 volumes 100% ice cold Ethanol.
3. Precipitate nucleotides at −20° C. overnight (or minimum 4 hours).
4. Centrifuge the samples at 14K rpm for 30 minutes at 4° C.
5. Carefully remove the supernatant and wash the pellet with 1 mL 70% Ethanol without disturbing the pellet.
6. Centrifuge the samples at 14K rpm for 15 minutes.
7. Carefully remove the Ethanol and allow pellet to air dry. Speed Vac is optional but do not overdry the pellet.
8. Rehydrate the pellet with 10 uL Nuclease-Free Water or Resuspension buffer (10 mM Tris, pH 8.3).

### Example 2

### Intramolecular Ligation

### Step 1-3′ Adapter Ligation

### Materials:

0.05-25 μM 3′ adapters (Bioo Scientific)
10×Mth RNA Ligase buffer (Bioo Scientific)

### 0.5-5000 U Mth RNA Ligase (Bioo Scientific)

RNA (1-10 μg of total RNA or isolated small RNA) (Bioo Scientific Cat. #5155-5182)

### 0%-50% PEG (MW 4000/8000)

### RNase Inhibitor

### Nuclease-Free Water (Bioo Scientific Cat. #801001)

1. Combine the following separately for EACH AIR Barcoded adapter:

| | | |
|---|---|---|
| 2-3 | μL | RNA (total RNA or isolated small RNA may be used |
| 1 | μL | 0.05-25 μM 3′ adapter |
| 1 | μL | 10X Mth RNA Ligase buffer |
| 2.4 | μL | 0-50% PEG (MW 4000/8000) |
| 1 | μL | RNase Inhibitor |
| 2-3 | μL | Nuclease-Free Water |
| 10 | μL | TOTAL |

2. Heat at 70-95° C. for 30 seconds and immediately place on ice for 2 minutes
3. Add to each:
   2 μL 0.5-5000 U Mth RNA Ligase
4. Incubate at 37-75° C. for 1-2 hours
5. Heat inactivate at 85-95° C. for 15 minutes

### Step 2—Reverse Transcription—1^{st} Strand Synthesis

### Materials:

10×RT buffer (Bioo Scientific Cat. #521002)
MMuLV Reverse Transcriptase (Bioo Scientific Cat. #521002)/equivalent Reverse Transcriptase enzyme
12.5 mM dNTP (Bioo Scientific Cat. #370601)

### 100 mM DTT

### RNase Inhibitor

### Nuclease-Free Water (Bioo Scientific Cat. #801001)

3′ ligated RNA (20 μL) (from steps 1 and 2)
2-100 μM RT primer
1. Add 1 μL RT primer (2-100 μM) to 3′ ligated RNA reaction mix.
2. Incubate at 70° for 2 minutes then immediately place on ice.
3. Add to each reaction

| | |
|---|---|
| 1 μL | 10X RT buffer |
| 0.5 μL | 12.5 mM dNTP |
| 1 μL | 100 mM DTT |
| 0.5 μL | RNase Inhibitor |
| 5 μL | Nuclease-Free Water |
| 1 μL | MMuLV Reverse Transcriptase/equivalent Revese |
| | Transcriptase enzyme |

4. Incubate at 44° C.-55° C. for 1-2 hours.

### Step 3—RNAse Treatment, Intramolecular Ligation

### Materials:

### RNAse H (Bioo Scientific)

10×Mth RNA Ligase intramolecular ligation buffer (Bioo Scientific)

### 0.5-5000 U Mth RNA Ligase (Bioo Scientific)

### Nuclease-Free Water (Bioo Scientific Cat. #801001)

1^{st} strand synthesis product (20 μL)
1. Add 1 μL RNAse H to each 1^{st} strand synthesis reaction (20 μL).
2. Incubate at 37° C. for 30 minutes.
3. Add each reaction sample:

| | |
|---|---|
| 5 μL | Nuclease-Free Water |
| 3 μL | 10x Mth RNA Ligase intramolecular ligation buffer |
| 1 μL | Mth RNA Ligase |

4. Incubate at 37-75° C. for 1-2 hours.
5. Heat inactivate at 85-95° C. for 5 minutes.

### Step 4—Endonuclease Cleavage

### Materials:

10 U Endonuclease V or other endonuclease enzyme (Bioo Scientific)
10× Endonuclease V buffer or other endonuclease buffer (Bioo Scientific)

### Nuclease-Free Water (Bioo Scientific Cat. #801001)

Intramolecular ligation product (30 μL)
Add to each intramolecular ligation product (30 μL)

| | |
|---|---|
| 17 μL | Nuclease-Free Water |
| 2 μL | 10x Endonuclease V buffer |
| 1 μL | 10 U Endonuclease V |

Incubate at 37° C., 1-2 hours.
Heat inactivate at 65° C., 10-25 minutes.

### Step 5—Polymerase Chain Reaction Amplification

### Materials:

### 25 μM PCR Primer 1

### 25 μM PCR Primer 2

5× DuroTaq PCR Master Mix (Bioo Scientific Cat. #370201)/equivalent thermostable Polymerase enzyme

### Nuclease-Free Water (Bioo Scientific Cat. #801001)

Endonuclease V cleavage product (5-20 μL)
1. Add to each Endonuclease V cleavage product (5-20 pt)

| | |
|---|---|
| 38 μL | Nuclease-Free Water |
| 10 μL | 5x DuroTaq PCR Master Mix/equivalent |
| | thermostable Polymerase enzyme |
| 1 μL | PCR Primer 1 |
| 1 μL | PCR Primer 2 |
| 50 μL | Total reaction volume |

2. Amplify

| | | | |
|---|---|---|---|
| 30 | sec | 95° C.-98° C. | |
| 10-15 | sec | 95° C.-98° C. | Repeat 5-25 |
| 30-60 | sec | 55° C.-65° C. | cycles |
| 15-30 | sec | 72° C. | |
| 10 | min | 72° C. | |

### Step 6—Purification

### Materials:

### 5-15% TBE gel

1×TBE buffer
Low molecular weight ladder with loading dye
4. Load samples with loading buffer into a 5-15% TBE gel
5. Run at 200 volts for 30-60 min
6. Cut the band corresponding to ˜150 nucleotides in length (do NOT cut out the 120 nucleotide band)
   Step 7—cDNA purification

### Materials:

### 1×Tris-EDTA, pH 7.5 (TE)

### 3 M NaOAc

### 95% Ethanol

### 70% Ethanol

### Nuclease-Free Water

1. Shred the gel pieces and then soak them in 1×TE for atleast 3 hours or overnight at room temperature.
2. Add 1/40^{th} volume 3 M NaOAc, 1/100^{th} volume glycogen and 4 volumes 100% ice cold Ethanol.
3. Precipitate nucleotides at −20° C. overnight (or minimum 4 hours).
4. Centrifuge the samples at 14K rpm for 30 minutes at 4° C.
5. Carefully remove the supernatant and wash the pellet with 1 mL 70% Ethanol without disturbing the pellet.
6. Centrifuge the samples at 14K rpm for 15 minutes.
7. Carefully remove the Ethanol and allow pellet to air dry. Speed Vac is optional but do not overdry the pellet.
8. Rehydrate the pellet with 10 uL H₂O or Resuspension buffer (10 mM Tris, pH 8.3).

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method of producing a library comprising:
obtaining a population of RNA molecules;
ligating a 3′ adapter oligonucleotide containing RNA bases, DNA bases, and/or synthetic bases and/or modified and/or randomized bases to the 3′ end of the population of RNA molecules, wherein a thermostable ligase is used to catalyze the ligation reaction and wherein the 3′ adapter oligonucleotide ligation reaction is carried out at a temperature greater than 40° C.;
ligating a 5′ RNA oligonucleotide adapter to the population of RNA molecules, wherein a thermostable ligase is used to catalyze the ligation reaction and wherein the 5′ adapter oligonucleotide ligation reaction is carried out at a temperature greater than 40° C.; and
converting the population of 3′/5′ ligated RNA molecules to complementary DNA (cDNA) molecules using reverse transcription.

2. The method of claim 1, further comprising amplifying the cDNA molecules by polymerase chain reaction.

3. The method of claim 1, further comprising purifying the population of 3′/5′ ligated RNA molecules.

4. The method of claim 1, wherein the 3′ adapter oligonucleotide comprises RNA bases or DNA bases.

5. The method of claim 1, wherein the 3′ adapter oligonucleotide comprises modified bases.

6. The method of claim 1, wherein the thermostable ligase used to ligate a 3′ adapter oligonucleotide to the 3′ end of the population of RNA molecules is Mth RNA Ligase.

7. The method of claim 1, wherein the thermostable ligase used to ligate a 3′ adapter oligonucleotide to the 3′ end of the population of RNA molecules is Mth RNA Ligase mutant K97A, Mth RNA Ligase mutant K246A, Mth RNA Ligase single mutant of any amino acids associated with the adenylyltransferase Motifs I through V, Mth RNA Ligase double mutant of any amino acids associated with the adenylyltransferase Motifs I through V, or Mth RNA Ligase triple mutant of any amino acids associated with the adenylyltransferase Motifs I through V.

8. The method of claim 1, wherein the 3′ adapter oligonucleotide ligation reaction is carried out at a temperature of between about 37° C. and about 75° C.

9. The method of claim 1, wherein the 3′ adapter oligonucleotide ligation reaction is carried out for a time effective to carry out the 3′ adapter oligonucleotide ligation reaction to completion.

10. The method of claim 1, wherein the population of RNA molecules comprises small RNAs ranging in size from about 15 bases to about 100 bases.

11. The method of claim 1, wherein the 3′ adapter oligonucleotide ligation reaction is carried out using a ligase reaction buffer comprising magnesium chloride, wherein the concentration magnesium chloride in the ligase reaction buffer is between about 1 mM to about 50 mM.

12. The method of claim 1, wherein the 3′ adapter oligonucleotide ligation reaction is carried out using a ligase reaction buffer comprising magnesium chloride, wherein the concentration magnesium chloride in the ligase reaction buffer is between about 10 mM to about 30 mM.

13. The method of claim 1, wherein the 3′ adapter oligonucleotide ligation reaction is carried out using a ligase reaction buffer comprising:
magnesium chloride at a concentration of between about 1 mM to about 50 mM;
dithiothreitol at a concentration ranging from about 1 mM to about 50 mM; and
Tris-HCl at a concentration ranging from about 1 mM to about 100 mM;
wherein the pH of the ligase reaction buffer is between about 5 to about 10.

14. The method of claim 1, wherein the 3′ adapter oligonucleotide is an adenylated oligonucleotide.

15. The method of claim 1, wherein the 3′ adapter oligonucleotide ligation reaction is performed in the presence of polyethylene glycol having a molecular weight of between about 4000 to about 8000.

16. A method of producing a library comprising:
obtaining a population of RNA molecules and/or DNA molecules;
ligating a 3′ adapter oligonucleotide to the 3′ end of the population of RNA molecules and/or DNA molecules, wherein a thermostable ligase is used to catalyze the ligation reaction and wherein the 3′ adapter oligonucleotide ligation reaction is carried out at a temperature greater than 40° C.;
converting the population of 3′ ligated RNA molecules and/or DNA molecules to complementary DNA (cDNA) molecules using reverse transcription;
intramolecularly ligating the resulting cDNA products; and
cleaving the resulting intramolecularly ligated cDNA using a targeted single-stranded DNA endonuclease to form linearized cDNA products.

17-32. (canceled)
